(19) United States
(12) Patent Application Publication
(10) Pub. No.: US 2024/0328940 A1
(43) Pub. Date: Oct. 3, 2024

(54) SYSTEM AND METHOD FOR MEASURING A PROPERTY OF A GAS IN A CONTAINER

(71) Applicant: GASPOROX AB, Lund (SE)

(72) Inventors: Johan AXELSSON, Lund (SE); Märta LEWANDER XU, Lund (SE); Rikard WELLANDER, Annelov (SE); Roland KOCH, Boswil (CH); Malin JONSSON, Lund (SE); Mikael SEBESTA, Dalby (SE); Patrik LUNDIN, Harlosa (SE); Anders LÅNGBERG, Veberod (SE)

(21) Appl. No.: 18/698,650

(22) PCT Filed: Oct. 4, 2022
(86) PCT No.: PCT/EP2022/077616

§ 371 Date: Apr. 4, 2024

(30) Foreign Application Priority Data
Oct. 5, 2021 (SE) 2151221-5

(51) Int. Cl. G01N 21/39; G01N 33/00

(52) U.S. Cl. CPC G01N 21/39; G01N 33/0027; G01N 2021/399

(57) ABSTRACT

A method and system for performing a measurement in-line for determining at least one property of a gas in a closed container by determining a position of the closed container in relation to an optical sensor using an intensity profile.

### BACKGROUND OF THE INVENTION

### Field of the Invention

This disclosure pertains to determining a property of a gas in a closed container by performing optical measurements. The properties may relate to a pressure and/or gas composition inside the container. Especially, the disclosure relates to a triggering method for establishing that the closed container is in a beam path of a light sensor used for measuring the property of the gas.

### Description of the Prior Art

When performing optical measurements on a gas in closed containers, it is important to use the right data for the estimation of the properties to achieve high sensitivity and accuracy.

For inline measurements, such as on a conveyance band, to obtain the right data, a starting point of when to start recording the data to be used for determining the properties of the gas and also when to stop the recording is required.

A separate trigger device for detecting a position of a container to be measure is commonly used for the purpose of determining when to start and to stop the recordings. When the triggering device detects a container at the position where the measurements are performed, the triggering device triggers the measurement device used for performing the optical measurement on the container.

A method to obtain a starting point and an end point in an intensity profile for measuring on containers has been described in WO 2016/051341. In this disclosure, a separate trigger device is used, the positioning sensor used for determining a position of the container and to trigger the laser measurements. In this disclosure, a time period for recording a spectrum is first determined based on a positioning sensor (a separate trigger device) and the speed of the container. The intensity profile is then recorded over the time period during which the bottle is passing a laser beam used for obtaining the intensity profile. After the intensity profile has been recorded, minima in the intensity profile, due to low transmission through the walls of a container entering and leaving a detection area, are determined and a sub-window in the intensity profile between the minima are determined and used for calculating a gas pressure in the container.

This method has some drawbacks, for example does the method require that the whole intensity profile is recorded before the minima can be found. Further, the method relies on additional sensor for the position of the container.

Hence, an improved system and method for triggering an optical measurement of a gas in a container would be advantageous. Preferably if the system may be made smaller and faster and adaptable for various sizes of containers, which all are advantageous for in-line measurements.

### SUMMARY OF THE DISCLOSURE

Accordingly, embodiments of the present disclosure preferably seek to mitigate, alleviate or eliminate one or more deficiencies, disadvantages or issues in the art, such as the above-identified, singly or in any combination by providing a system or method according to the appended patent claims for non-destructively determining a property of a gas in a closed container.

A first aspect of the disclosure relates to a method of performing a measurement in-line for determining at least one property of at least one gas in a closed container, the closed container includes a measuring area with a diameter. The method may include emitting a light beam between a light source and a detector, wherein the light beam may be transmitted through a detection area. The light beam may have a wavelength tunable with an absorption wavelength of the gas.

The method may also include obtaining a transmission signal from the detector related to the light beam transmitted through the detection area.

Further, the method may also include advancing the closed container towards the detection area at a speed.

Also, the method may include determining, based on analysis of the transmission signal, when the closed container is entering the detection area.

The method could also include estimating the at least one property of the at least one gas based on the transmission signal related to the light beam transmitted through the measuring area of the closed container while the closed container is advancing through the detection area.

In some examples, the method may include continuously obtaining the transmission signal.

In some examples, the method may include using the light beam both for determining when the closed container is entering the detection area and for estimating the at least one property of the gas in the closed container.

This means that the same sensor is used both for determining when the closed container is entering the detection area and for estimating the at least one property of the gas in the closed container.

In some examples, the method may include that the at least one property of the gas includes at least one of a gas pressure in the closed container and/or a concentration of the gas in the closed container.

In some examples, the method may include that the transmission signal is used for determining a rise (increase) and/or a fall (decrease) in an intensity of the transmission signal due to the closed container entering the detection area and/or exiting the detection area.

In some examples, the method may include that a derivative of the intensity is used for determining the rise and/or the fall in the intensity of the transmission signal.

In some examples, the method may include that the light beam is pulsed, and each pulse has an amplitude, and wherein the pulse amplitude is used for determining the rise and/or fall of the intensity of the transmission signal.

In some examples, the method may include that the detector is a PSD and wherein the transmission signal is used for determining a deflection of the light beam to detect when the closed container is entering and/or exiting the detection area.

In some examples, the method may include that the deflection is used for determining a rise (increase) and/or a fall (decrease) in an intensity of the transmission signal due to the closed container entering the detection area and/or exciting the detection area.

In some examples, the method may include that the speed, together with the transmission signal, is used for determining a starting point and/or an ending point of a time period for obtaining data to be used for estimating the at least one property of the gas in the closed container.

In some examples, the method may include that the starting point and/or ending point of the time period for is set at a time period, based on the speed, after it has been determined that the closed container has entered the detection area based on the transmission signal.

In some examples, the method may include that the diameter of the measuring area of the closed container is used to determining the ending point of the time period used obtaining data to be used for estimating the property of the gas in the closed container.

In some examples, the method may include that a time measured between two of the fall and/or the rise, determined based on the intensity of the transmission signal, is used for estimating the speed and/or the diameter of the measuring area of the closed container.

In some examples, the method may include that the measuring area of the closed container is part of a head space.

In some examples, the method may include that the transmission signal is used for determining a background concentration of the gas in the detection area when the closed container is not in the detection area.

In some examples, the method may include that the background concentration of the gas is used for reducing an offset when performing an estimation of the property of the gas in the closed container.

Another aspect of the disclosure relates to a system for performing a measurement in-line for determining at least one property of a gas in a closed container, the closed container comprises a measuring area with a diameter. The system may include an optical sensor configured for emitting a light beam between a light source and a detector of the optical sensor, wherein the light beam is transmittable through a detection area. The light beam may have a wavelength tunable with an absorption wavelength of the gas in the closed container.

The sensor may be configured for obtaining a transmission signal related to the light beam.

The system may also include means for advancing the closed container towards the detection area at a speed.

The system could also include a control unit configured for determining, based on the transmission signal, when the closed container is entering the detection area. The control unit may further be configured for estimating the at least one property of the at least one gas based on the transmission signal related to the light beam transmitted through the measuring area of the closed container while the closed container is advancing through the detection area.

The system may also include a control unit configured for determining, based on the transmitted light signal being detected, the integrity of said container.

### BRIEF DESCRIPTION OF THE DRAWINGS

These and other aspects, features and advantages of which examples of the disclosure are capable of will be apparent and elucidated from the following description of examples of the present disclosure, reference being made to the accompanying drawings, in which:

FIG. 1 is illustrating a schematic example of a container being measured using a system of the disclosure;

FIG. 2 is illustrating a schematic example of a measured intensity while a closed container passes a light beam;

FIG. 3 is illustrating schematic examples of light pulses;

FIG. 4 is illustrating a schematic example a flow-chart for a method for measuring according to the disclosure;

FIG. 5A is illustrating a schematic carousel for moving containers; and

FIGS. 5B and 5C are illustrating measurements performed on containers arranged in the carousel illustrated in FIG. 5A.

### DESCRIPTION OF EXAMPLES

Specific examples of the disclosure will now be described with reference to the accompanying drawings. This disclosure may, however, be embodied in many different forms and should not be construed as limited to the examples set forth herein; rather, these examples are provided so that this disclosure will be thorough and complete, and will fully convey the scope of the disclosure to those skilled in the art.

The following disclosure focuses on examples of non-destructive testing of closed containers. The present disclosure is applicable to determining the property of a gas in a closed container. The closed container is at least partially made from an optically transparent material. The closed container may be a bottle, a vail, a jar, an ampoule, a can etc. The closed container may also be packages, bags, trays etc. The containers may be completely filled by at least one gas. In some examples, the closed container may be at least partially filled with a content, such as a liquid or a solid. When a container is partially filled with a content, the container may include a gas filled headspace.

Determining the property of a gas in a closed container may provide useful information, for example may such measurements be useful to determine an integrity of the container. An integrity of a closed container may provide information about the container being properly sealed or is if the container is leaking. The property may also be able to provide information of the content in the container.

The property may involve obtaining a gas pressure and/or determining a gas, such as a gas concentration, in the closed container.

A container may be a closed bag or closed tray that includes at least one species of a gas, Examples may be containers having a modified atmosphere (MAP). Modified atmosphere is commonly used in packages in order to improve the shelf life, for example in food packages, drugs, etc. gases commonly used are carbon dioxide (CO2) or nitrogen (N2) to lower the amount of oxygen (O2). This is made in order to slow down growth of aerobic organisms and prevent oxidation reactions. Hence it is important to monitor these packages and make sure that there is no leakage, for example during packaging. Apart from carbon dioxide (CO2), and oxygen (O2), other gases can be monitored as well, depending on the container and the product.

The disclosure is in particular directed to a method for determining when the closed container is in the beam path and the measurements may be conducted.

The method described herein has the benefit, that it can be done with a mechanically less extensive setup.

The pressure and gas composition inside the container may both be measured using optical absorption spectroscopy. Especially the method tuneable diode laser absorption spectroscopy (TDLAS) may be applied.

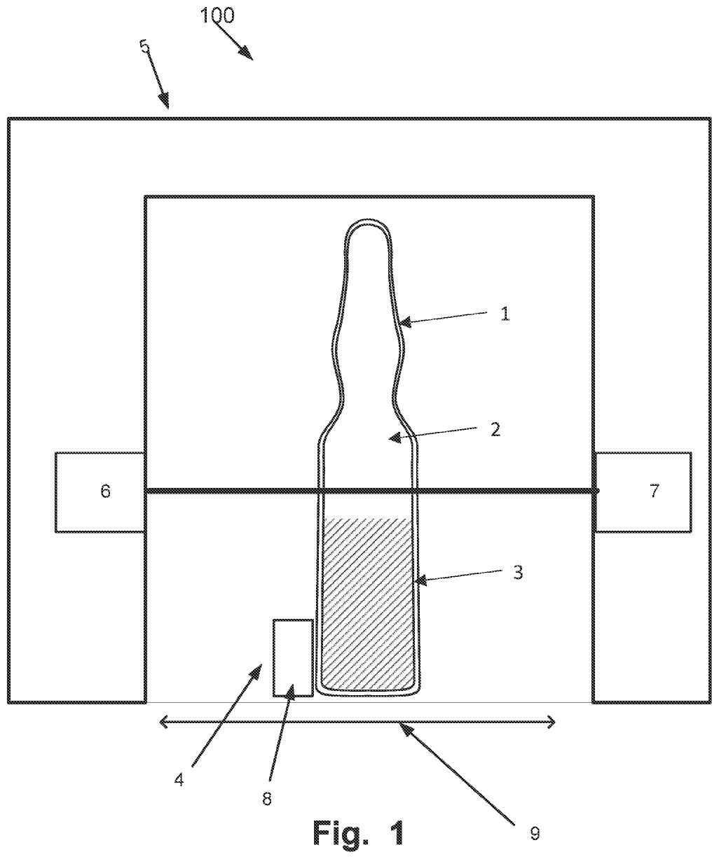

FIG. 1 is illustrating a schematic example 100 of system. The system is designed for optical measurements to estimate a property of a gas 2 in a container 1. The container may include a content 3 and a gas filled headspace 2. The container is illustrated as being arranged in a detection area 4.

The detection area 4 may be defined by a partial enclosure 5, such as two opposite walls, a tunnel or a walled space. A walled space may, for example, be arranged as an arch or have a shape of an inverted U.

The detection area 4 may include an optical sensor 6, 7. The optical sensor 6,7 may be arranged at opposite walls of the partial enclosure 5. The optical sensor is configured to emitting a light beam to be transmitted through the detection area 4 between a light source 6 and a detector 7. Alternatively, the light source 6 and the detector 7 are arranged at the same side and a reflecting member is arranged at the opposite side. The reflecting member may be diffusing the light.

The sensor 6, 7 may be configured for measuring on a headspace 2 of the container 1. Preferably, the sensor 6, 7 is designed or adjusted to detect the spectroscopic signal of at least one of the gases that are present inside the container 1.

The light beam to be transmitted through the detection area 4 may have a wavelength tunable with an absorption peak of a gas in the container.

The illustrated arrangement could be adapted to perform the inspections in-line. For example, by having the beam crossing a convey belt moving the containers.

The container 1 may be transported on a conveyance band (not illustrated) or be arranged in a carousel (not illustrated) for moving the container through the detection area 4. Conveyance bands or carousels are known in the art for in-line use during filling and sealing of containers.

The container 1 has a certain amount of gas and may be subjected to an integrity test. In case there is a leak in the container, the gas inside the container may leak out into the surrounding and/or gas in the surrounding may leak into the container. Thus, a concentration of the gas inside the container 1 may change, such as an absolute concentration, as may the pressure inside the container 1.

In the described system, the light source 6 may be a white light source, for example transmitting a collimated light beam, or at least one laser source, such as a diode laser, a semiconductor laser. The wavelengths or wavelength range used for 5 the light source is selected to match the absorption spectra of at least one species of the gas inside the container. The detector 7 may be, for example, a photodiode, a photomultiplier, a CCD detector, a CMOS detector, a Si detector, an InGaAs detector, selected to be able to detect the wavelengths or wavelength range of the light source. The detector 7 may also be an array, such as a position sensitive device (PSD).

The detected light may be analysed in a control unit (not shown) for determining the property of the at least one gas in the container 1. The control unit may be a computer, a microprocessor or an electronic circuit that could run code, or a software configured for analysing the light detected by the detector.

By detecting the at least one gas inside the container 1, it is possible to determine the pressure inside the container 1 and/or a concentration of the at least on gas inside the container 1 based on the detected transmitted signal. The measured pressure inside the container 1 and/or a concentration of the at least on gas inside the container 1 can be used to determine the integrity of the container 1.

In some examples, the optical sensor 6,7 consists of a sensor based on tunable diode-laser absorption spectroscopy (TDLAS).

In some examples, the optical sensor 6,7 consists of a sensor for gas in scattering media absorption spectroscopy (GASMAS). The GASMAS technique may be used for investigating sharp gas spectral signatures, typically 10000 times sharper than those of the host material, in which the gas is trapped in pores or cavities, such as headspaces 2 of a container 1. GASMAS combines narrow band diode laser spectroscopy, developed for atmospheric gas monitoring, with diffuse media optical propagation, well known from biomedical optics. Photons injected into a container 1 from a narrow band optical source may be detected in transmission or in backscattering arrangements. The technique has also been extended to remote sensing applications (LIDAR GASMAS or Multiple Scattering LIDAR. One example of a GASMAS sensor system and detection principle is described in EP 10720151.9 (Svanberg et al.) which is herein incorporated by reference.

The gas sensing instrument described in EP 10720151.9 consists of two diode lasers drivers for monitoring oxygen and water vapour inside a container. Monitoring of other gases or more than two gases are possible depending on the wavelengths used. The light from the diode lasers (DLs) is brought together and separated into two fibres-one used to monitor the background and one sent to the sample. The two diode lasers may operate at the wavelengths were the container 1 is translucent, making the GASMAS technique suitable. The laser light is guided to the headspace via optical fibres and a hand-held fibre head. The scattered light emerging out from the sample is acquired by a detector and the generated signal is sampled by a computer (not illustrated). In this example, wavelength modulation techniques are used to increase the sensitivity of the instrument by sinusoidally modulating the wavelength and studying the generated harmonics. In some examples, simultaneous detection of water vapour and oxygen is enabled by modulating at different frequencies.

The system 100 may assess the containers 1 without contacting the containers 1 and instead detect the gas inside the containers from a remote distance. This is advantageous as the speed of detection may be increased and also for inline monitoring of containers.

The method described in EP 10720151.9 comprises emitting light from a narrow-band laser source towards the container from outside of the container. Measuring an absorption signal of the light scattered in the container, the absorption caused by at least one gas in the container when the light is scattered and travels in the container. The measuring is made outside of the container, and the assessment is non-intrusive with regard to the container.

Due to the scattering of the light in the sample a complication at the evaluation of the absorption signals obtained with the GASMAS method is the unknown gas interaction path length which the light has experienced.

The path length is important in traditional gas absorption spectroscopy for concentration quantification, as determined by the Beer-Lamberts law.

Other types of GASMAS systems and methods are described in the article “Optical Analysis of Trapped Gas-Gas in Scattering Media Absorption Spectroscopy”; Svanberg, S; Laser Physics, 2010, Vol. 20, No. 1, pp. 68-77; ISSN 1054-660X, these systems and methods described therein are incorporated by reference.

For example, oxygen and water vapour may be monitored simultaneously in transmission mode. Monitoring of other gases or more than two gases are possible depending on the wavelengths used. Alternatively, in some examples, the system 400 may be arranged for backscattering measurements. A common detector is used, and the two signals are separated by phase-sensitive detection of the two spectroscopic signals, tagged with different modulation frequencies. Partial common fibre optical pathways may be used. The GASMAS signal, which is recorded in, for example, arrangements such as those just described depends on the gas concentration in pores or headspaces, the gas, and on the effective path length through gas in the complex multiple scattering process. The strength of the recorded gas imprint is therefore generally expressed as an equivalent path length, Leq.

Alternatively, the mean path length through the scattering medium may be derived from time resolved measurements. Delayed coincidence single photon counting techniques may be used to obtain the histogram of photon arrival times.

In some examples, the optical sensor 6, 7 is working in transmission mode, i.e., the light transmitter 6 is located on one side of a detection area 4, and the light detector 7 is located on the opposite side of a headspace 2 of the container 1, and a light beam is transmitted from the light transmitter 6 through the container, such as through a headspace 2, to the light detector 7.

In some examples, the optical sensor is working in reflection mode, i.e., the light transmitter is located on the same side of the container as the light detector, and the light detector 7 records back-scattered light from the container 1.

In some examples, the light transmitter 6 and the light detector 7 are positioned in arbitrary positions in relation to each other on the container 1, and the light detector 7 records scattered light from the container 1.

In some examples, the light is guided to and/or from the container by means of optical fibres. In some examples, the light is guided to and/or from the container via optical components including lenses, mirrors, windows, or other means of guiding and directing light.

In some examples of a detection area 4, such as a partial enclosure 5, the distance between the opposite walls where the optical sensor 6,7 is arranged, may have a fixed distance 9. The fixed distance 9 may be selected to allow most common sizes of containers to be pass through the detection are 4. For bottles and vials, the distance 9 may be less than 10 cm, such as less than 9 cm, such as less than 8 cm, such as 5 cm.

When a container is passed through the detection area 4 it may be an advantage if the container is close to the detector, such as less than 1 cm. This may be achieved by how the containers are arranged on the conveyance band or carousel in relation to the detection area 4, such as the partial enclosure 5. Alternatively, in some examples of the system 100, a guider 8 may be used to arrange the container 1 closer to the detector 7 by guiding the container towards the detector 7.

Should the properties of the content in the vial not match an expected value or be within an expected value range, the vial may be rejected using a rejection system. The rejection system could either be mechanical using a mechanicals pusher to push to vial off the conveyance band. Alternatively, the rejection system may use compressed air to push the vial off the conveyance band.

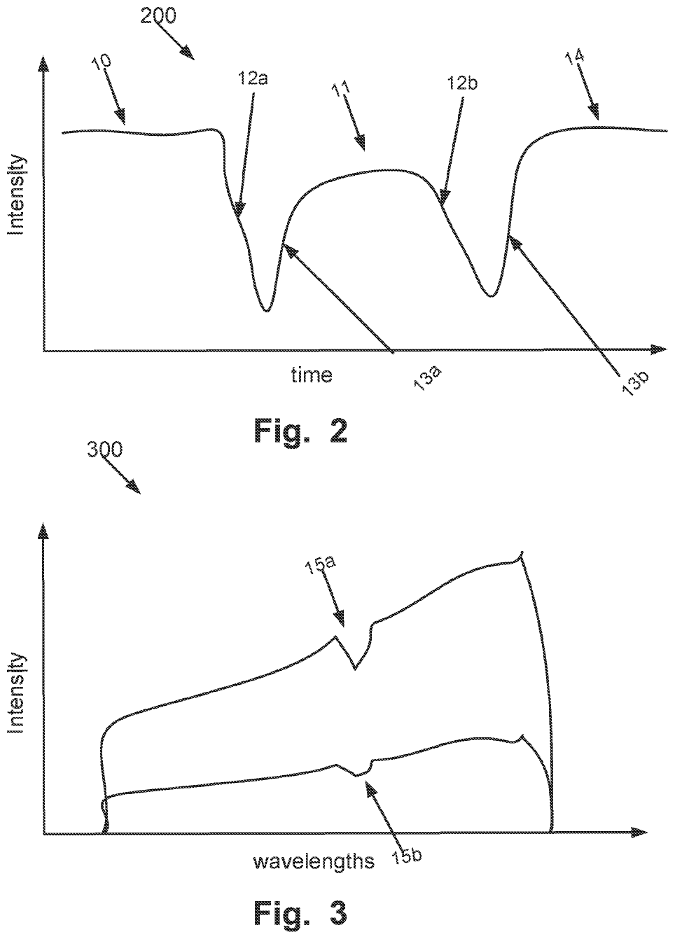

FIG. 2 is illustrating a schematic example of a measured intensity profile 200 while a closed container passes a light beam. When a closed container passes a light beam, the detector may detect a transmission signal that generates an intensity profile. Part of this intensity profile may be used for determining a property of a gas in the closed container. The light beam may be pulsed and the property of the gas inside the container may be obtained by sum-up the pulses over certain time period while the laser beam is transmitted through the container.

It is therefore important to establish when to start collecting data for estimating the property of the gas in the container and when to stop. In the intensity profile 200, this period is 11 and is found between two distinct minima. The minima are obtained in the intensity profile 200 when walls of the container pass the light beam.

To establish when to start collecting data to be used for estimating the property of the gas in the container, a fall (decrease) 12a and/or rise (increase) 13a in the intensity profile may be detected. One way of finding the fall 12a and/or the rise 13a may be to obtain a derivative of the intensity. This may be done by averaging the transmission signal from the optical sensor over time and then obtaining the derivative.

By knowing the speed the closed container is advanced through the light beam, and some characteristics of the container, such as the width or diameter of the portion of the container to be measured, the start point and end point for collecting the transmission signal to be used for the estimation of the property of the gas in the container can be established. For example, if the fall 12a in the intensity profile is detected, by knowing the speed of the conveyance band or carousel advancing the container, a starting point for collecting data at the rise 13a can be established, such as close to the beginning of the intensity period 11. A stop point for collecting the date at the fall 12a may be established by knowing the speed and the with and/or diameter of the container, such as a stop point close to the end of the intensity period 11.

In some examples, a separate sensor may be used for measuring the speed of the conveyance band. For example, the speed of the conveyance band may be measure continuously. The measured speed may be used to adjust the speed of the conveyance band continuously and fast without any delays, such as correcting variations in the speed of the conveyance band to a pre-set speed. Alternatively, and/or additionally, the sensor is an encoder and the data from the encoder may be used to continuously adjust the speed value used for calculating the properties of the gas inside the vial.

In this way the same light beam can be used both for triggering the measurement and for conducting the measurement. Further, the collection and analysis of the collected data to estimate a property of the gas in the closed container may be run faster than if the intensity spectrum has to be analysed for the right area to be used for the measurement after the whole intensity profile has been recorded.

Alternatively, to using the same laser for both triggering of the measurements and for performing the measurement. A separate positioning sensor may be used for triggering the collection of the data. For example, a second laser beam may be used for detecting when the container enters the detection area and/or leaving the detection area.

Alternatively, and/or additionally, in some examples, the fall 12a and rise 13a may be detected by using the amplitude of the pulses in the light beam. This may indicate the container entering the detection area. When the pulses of the light beam are lower than a threshold they will be discarded. This may indicate that there is a fall 12a in the intensity profile when the intensity increases over the threshold again, a rise 13a in the intensity profile may be indicated. Again, by knowing the speed of the conveyance band or carousel advancing the container, a starting point for collecting data at the rise 13a may be established as well as a stop at the fall 12a which may be established by knowing the speed and the with or diameter of the container.

In some examples, instead of having to know the speed the closed container is moving, both the points when the container enters the detection area and leaves the detection area may be determined from the intensity data. In this case, entry is established using any of the techniques described previously. The exit point may be derived by determining the fall 12b or rise 13b representing the second minimum after intensity period 11 in the intensity profile illustrated in FIG. 2. The second minimum may be determined using any of the previously described techniques herein.

Alternatively, and/or additionally, in some examples, the fall 12a, 12b and rise 13a, 13b may be detected by using a PSD to measure the position of the light being transmitted through the detection area. This may be done by observing the deflection of the light beam as the light hitting the walls of the container will have a different angle compared to the transmission signal being transmitted through the container, the minimums in the intensity profile may be established.

Additionally, in some examples, to obtain data that has a certain quality for the estimation of a property of the gas in the closed container, a threshold for the pulsed amplitude may be used. When the amplitude of the pulses is low, the absorption feature will be small, thus carry little information. It is therefore preferred that the pulses used for the estimation of the property of the gas is collected mainly during the intensity period 11. This is the period when the intensity of the transmission signal through the container is at its highest.

For example, the first minimum before the intensity period 11 of the intensity profile 200 is determined and the container is considered to entering the detection area. This means that the data may be collected for the estimation of the property of the gas inside the closed container. The data initially collected, may not have the amplitude required to obtain a good estimation of the property of the gas. A threshold may therefore be set and any pulses having an amplitude lower than the threshold will be discarded. When the container moved through the detection area, the pulses will change it characteristics and the amplitude will increase. When the amplitude reaches the set threshold value, the intensity period 11 has started and the pulses may no longer be discarded.

When the value of the amplitude of the pulses of the light beam fall under the set threshold value the pulses may be disregarded again. This may happen when the container has moved through the detection area and reached the end of the intensity period 11. The collection of data may continue until the second minimum has been determined.

The thresholds may also be used for disregarding intensities during the intensity period 11 that falls below the threshold. This may for example be due to dirt on the vial or irregularities in the glass walls of the vials, such as the thickness of the glass walls of the vial not being consistent.

By detecting at least two fall points 12a, 12b and/or rise point 13a, 13b, the speed of the conveyance band or carousel advancing the container may be estimated. For example, the speed may be obtained by establishing the fall 12a, 12b and rise 13a, 13b of one of the minima of the intensity profile. Alternatively, speed may be obtained by establishing the fall 12a or rise 13a of the first minimum and the fall 12b or rise 13b of the second minimum. Alternatively, the speed may be obtained by analysing the derivative or second derivative of the intensity profile, during the rise or fall.

This may be used for monitoring the speed and adjust for variation in the speed when performing the calculations or to control the speed of the conveyance band or carousel advancing the container.

In some examples, should the monitored speed differ from the set speed of the conveyance band, this may have an effect on the accuracy of the measurements on the gas inside the vial since the portion of the container to be measured on may in some examples be determined using the speed of the conveyance band. The time period from which the intensity for the calculation is taken depends on the movement of the vial in and out of the detection area. The calculation may be corrected using the monitored speed, thus improve the accuracy of the measurements.

In some examples, should the speed differ from the set speed, the monitored speed may be set in the software so that the next vial may be analysed based on the previously measured speed.

Also, by establishing the first rise 13a and the second fall 12b in the intensity profile, the width or diameter of the container may be established. This information may be used for adjusting for variation in sizes of the containers or to establish if a new type of container has been introduced into the system.

The intensity between containers 10, 14 may be used for calculating a concentration of the gas in the surrounding or the background. This may, for example be ambient air or a controlled gas composition in the detection area. This value may be averaged and used to remove or reduce an offset in the estimation of a concentration of a gas in the closed container the in those cases the container has a smaller diameter than the distance between the light source and detector of the sensor, or the distance between the sensor and a reflecting surface opposite the sensor, in the detection area.

Alternatively, the value obtained for the gas in the period 10 prior to the period 11 is used for removing or reducing an offset due to surrounding gas when estimating the property of the gas in the container

The intensity between containers 10, 14 may also be used to determine if the containers are in phase as the intensity between containers 10, 14 should differ from the intensity 11 measured through the container. Should the containers not be in phase, the measurements for estimating a property of the gas in the closed containers, may be performed on the wrong section of the intensity profile, such as between the containers.

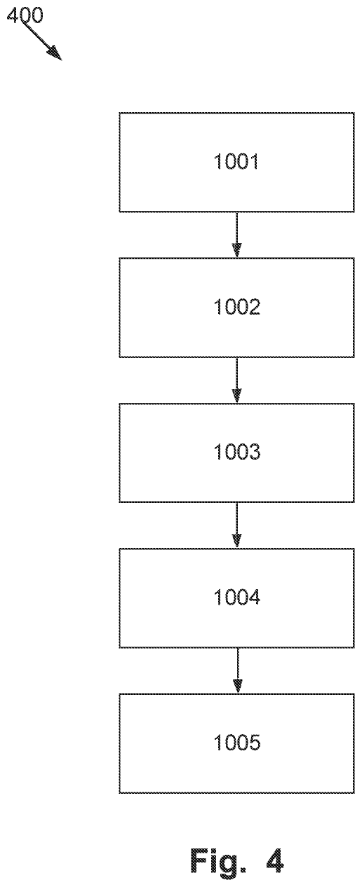

FIG. 3 is illustrating schematic examples of light pulses 300. Each light pulse includes an absorption peak 15a, 15b related to the gas in the container. By weighting the pulses, the pulses closer to and/or on the first rise 13a in the intensity profile and the second fall 12b in the intensity profile will have less impact on the estimation of the property of the gas in the container. Alternatively, when sum the pulses up over the section 11, the pulses closer to and/or on the first rise 13a in the intensity profile and the second fall 12b in the intensity profile will have less impact on the estimation of the property of the gas in the container.

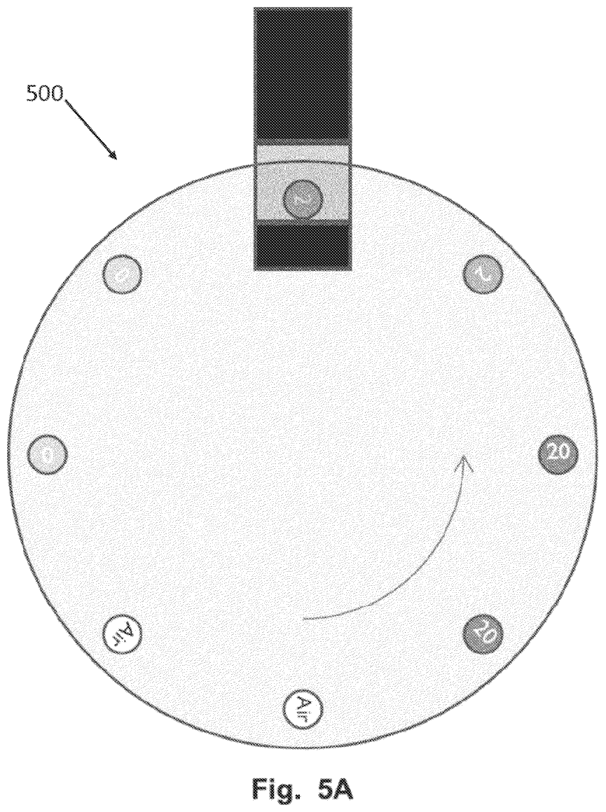

FIG. 4 is illustrating a schematic example a flow-chart for a method 400 for measuring a property of a gas in a container according to the disclosure. The method may include:

Emitting 1001 a light beam between a light source and a detector. The light beam may be transmitted through a detection area. The light beam may have a wavelength tunable with an absorption wavelength of the gas.

Obtaining 1002 a transmission signal from the detector related to the light beam transmitted through the detection area.

Advancing 1003 the closed container towards the detection area at a speed.

Determining 1004, based on analysis of the transmission signal, when the closed container is entering the detection area.

Estimating 1005 the at least one property of the at least one gas based on the transmission signal related to the light beam transmitted through the closed container while the closed container is advancing through the detection area.

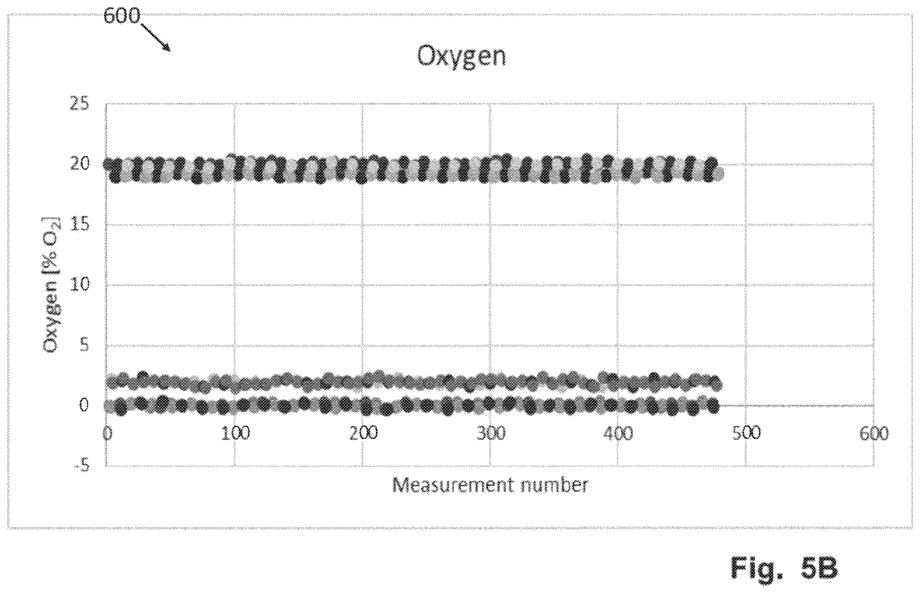

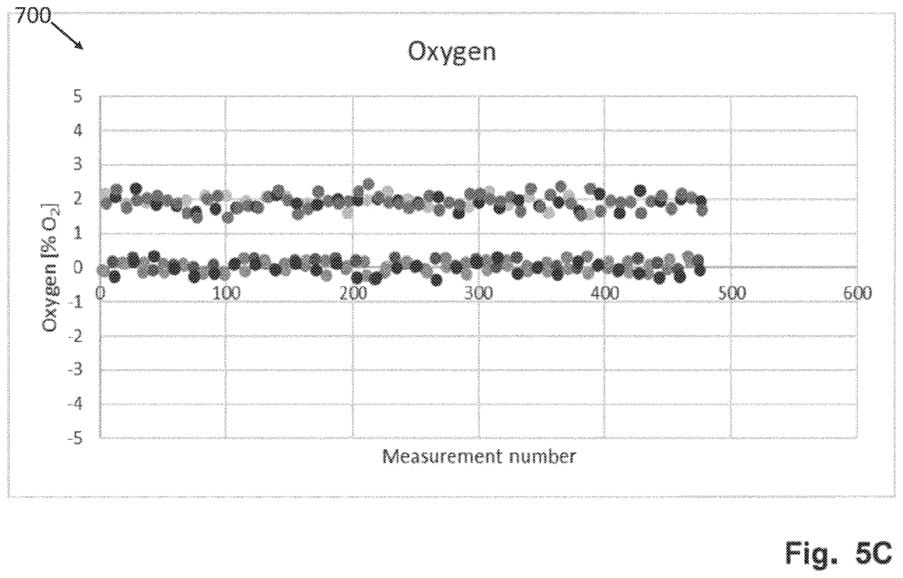

FIG. 5A is illustrating a schematic carousel 500 for moving containers used for testing the method herein. The positions in the containers included 2 vials with 0% oxygen, 2 vials with 2% oxygen, 2 vials with 20% oxygen and 2 vials with air.

FIGS. 5B and 5C are illustrating oxygen concentration measurements 600, 700 performed on containers arranged in the carousel illustrated in FIG. 5A. The measurements are illustrating how accurate the measurements and that it is possible to detect a difference both between the vials having 0% oxygen and 2% oxygen but also between the vials having 20% oxygen and air.

The present invention has been described above with reference to specific examples. However, other examples than the above described are equally possible within the scope of the disclosure. Different method steps than those described above, performing the method by hardware or software, may be provided within the scope of the invention. The different features and steps of the invention may be combined in other combinations than those described. The scope of the disclosure is only limited by the appended patent claims.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

1. A method of performing a measurement in-line for determining at least one property of a gas in a closed container, said closed container comprises a measuring area with a diameter, said method comprising:
emitting a light beam between a light source and a detector, wherein said light beam is transmitted through a detection area; said light beam has a wavelength tunable with an absorption wavelength of said gas;
obtaining a transmission signal from the detector related to said light beam transmitted through said detection area;
advancing said closed container towards said detection area at a speed;
determining, based on analysis of said transmission signal, when said closed container is entering said detection area;
estimating said at least one property of said at least one gas based on said transmission signal related to said light beam transmitted through said measuring are of said closed container while said closed container is advancing through said detection area.

2. The method of claim 1, comprising continuously obtaining said transmission signal.

3. The method of claim 1, comprising using said light beam both for determining when said closed container is entering said detection area and for estimating said at least one property of said gas in said closed container.

4. The method of claim 1, wherein said at least one property of said gas includes at least one of a gas pressure in said closed container and/or a concentration of said gas in said closed container.

5. The method of claim 1, wherein said transmission signal is used for determining a rise and/or a fall in an intensity of said transmission signal due to said closed container entering said detection area and/or exiting said detection area.

6. The method of claim 5, wherein a derivative of said intensity is used for determining said rise and/or said fall in said intensity of said transmission signal.

7. The method of claim 5, wherein said light beam is pulsed and each pulse has an amplitude, and wherein said amplitude of said pulse is used for determining said rise and/or fall of said intensity of said transmission signal.

8. The method of claim 1, wherein said detector is a PSD and wherein said transmission signal is used for determining a deflection of said light beam to detect when said closed container is entering and/or exiting said detection area.

9. The method of claim 8, wherein said deflection is used for determining a rise and/or a fall in an intensity of said transmission signal due to said closed container entering said detection area and/or exciting said detection area.

10. The method of claim 1, wherein said speed, together with said transmission signal, is used for determining a starting point and/or an ending point for estimating said at least one property.

11. The method of claim 10, wherein said starting point and/or ending point is set at a time period, based on said speed, after it has been determined that said closed container has entered said detection area based on said transmission signal.

12. The method of claim 10, wherein said diameter of said measuring area of said closed container is used to determining said ending point.

13. The method of claim 4, wherein a time measured between two of said fall and/or said rise determined based on said intensity of said transmission signal is used for estimating said speed and/or said diameter of said measuring area of said closed container.

14. The method of claim 1, wherein said measuring area of said closed container is part of a head space.

15. The method of claim 1, wherein said transmission signal is used for determining a background concentration of said gas when said closed container is not in said detection area.

16. The method of claim 15, wherein said background concentration of said gas is used for removing an offset when performing an estimation of said property of said gas in said closed container.

17. A system for preforming a measurement in-line for determining at least one property of a gas in a closed container, said closed container comprises a measuring area with a diameter, said system comprising:
an optical sensor configured for emitting a light beam between a light source and a detector of said optical sensor, wherein said light beam is transmittable through a detection area; said light beam has a wavelength tunable with an absorption wavelength of said gas;
said sensor is configured for obtaining a transmission signal related to said light beam;
means for advancing said closed container towards said detection area at a speed;
a control unit configured for determining, based on said transmission signal, when said closed container is entering said detection area;
said control unit is further configured for estimating said at least one property of said at least one gas based on said transmission signal related to said light beam transmitted through said measuring are of said closed container while said closed container is advancing through said detection area.

18. The system of claim 17, wherein said system is configured for performing the method steps of claim 2.
